(19) United States
(12) Patent Application Publication
(10) Pub. No.: US 2005/0003449 A1
(43) Pub. Date: Jan. 6, 2005

(54) Method for determining molecular affinities for human serum albumin

(76) Inventors: Ronald Waldo Sarver JR., Dexter, MI (US); Atli Thorarensen, O'Fallon, MO (US)

Correspondence Address: PHARMACIA & UPJOHN, 301 HENRIETTA ST, 0228-32-LAW, KALAMAZOO, MI, 49007, US

(21) Appl. No.: 10/751,815

(22) Filed: Jan. 5, 2004

Related U.S. Application Data
(60) Provisional application No. 60/438,709, filed on Jan. 8, 2003.
(60) Provisional application No. 60/440,680, filed on Jan. 17, 2003.

(51) Int. Cl. G01N 33/53

(52) U.S. Cl. 435/7.1

(57) ABSTRACT

The invention features a fluorescent spectroscopic method for determining molecular affinities of test compounds for human serum albumin.

### CROSS REFERENCE TO RELATED APPLICATIONS

This application claims the benefit of the following provisional applications: Application Ser. No. 60/438,709 filed Jan. 8, 2003 and Ser. No. 60/440,680 filed Jan. 17, 2003 both under 35 U.S.C. 119(e)(1).

### FIELD OF THE INVENTION

This invention provides a new fluorescence based assay for determining molecular affinities of drug candidates for Human Serum Albumin.

### BACKGROUND OF THE INVENTION

Many drugs and some endogenous substances bind reversibly and with high affinity to human serum albumin. See, for example, Otagiri et. al. Biol. Pharm. Bull. 25, 695 (2002). Human Serum Albumin (HSA) has two major hydrophobic binding pockets, Site I on domain IIA and Site II on domain IIIA. Reversible binding to albumin extends the lifetime of a drug in plasma but it also decreases the concentration of the free drug available for physiological action. For this reason, it is important to know the affinity of drug candidates for albumin as a step in the drug discovery process. Thus, there is a need for a method for the rapid quantitation of the binding affinities of ligands toward albumin.

### SUMMARY OF THE INVENTION

In general, the invention features a fluorescent spectroscopic method for determining the molecular affinity of certain drug candidates for HSA. The method includes monitoring a fluorescent signal from a probe compound to determine the molecular affinities of certain drug candidates.

In one aspect, the invention features a method for determining the binding affinity of an analyte including the steps of:
a) providing a buffer solution;
b) adding an analyte to the solution;
c) adding to the solution; a probe compound which binds to a plurality of Human Serum Albumin sites.
d) adding Human Serum Albumin to the solution;
e) irradiating the solution containing the analyte, probe compound, and Human Serum Albumin;
f) measuring the fluorescence of the irradiated solution; and
g) calculating the binding affinity of the analyte based on the measured fluorescence.

In another aspect, the invention features a method of conducting a high-throughput screen. The method includes the steps of
a) providing a plurality of buffer solutions;
b) providing a plurality of analytes;
c) adding one analyte from the plurality to a plurality of the buffer solutions;
d) adding a probe compound which binds to sites I and II of Human Serum Albumin to a plurality of the solutions containing an analyte;
e) adding Human Serum Albumin to a plurality of the solutions containing an analyte, and the probe compound;
f) irradiating a plurality of the solutions containing the analyte, the probe compound, and Human Serum Albumin;
g) measuring the fluorescence of the irradiated solutions; and
h) calculating the binding affinity of the analyte in each of the solutions based on the measured fluorescence.

Each aspect of the invention may include one or more of the following. Calculating the binding affinity of the analyte includes determining the percent displacement of the probe. The percent displacement of the probe is determined via the equation
D=100−[(f₀−fₐ)/(f₀−f_{b})]*100,
where, D is the percent THE PROBE displaced from Human Serum Albumin by the analyte, f₀ is the fluorescence of THE PROBE in the buffer solution, f_{b} is the fluorescence of THE PROBE bound to HSA, and fₐ is the fluorescence of the solution containing the analyte and the probe compound. The fluorescence is measured between 480-580 nm. The method further includes measuring the fluorescence of the solution containing the probe compound and the analyte. The probe is a compound of Formula I 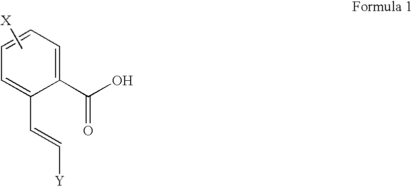
   wherein:
X is one to three substituents selected from the group of halogen, —CN, NO₂, aryl, —C(O)—R, in which R is an optionally substituted C₁-C₄ alkyl or an optionally substituted aryl; and Y is substituted or unsubstituted heteroaryl. Y may be an optionally substituted indolyl, such as 1-methyl-indol-2-yl. The probe may be 5-cyano-2-[(E)-2-(1-methyl-1H-indol-2-yl)ethenyl]benzoic acid.

Advantageously the probe compounds have a high quantum yield and an excitation wavelength longer than the test compounds' absorption bands. Unexpectedly, the probe compounds of this invention bind with high affinity in two distinctive sites within HSA, e.g., near site I and at site II. The probe compounds of the invention may be used for displacement studies with other less fluorescent compounds of similar structure and for high throughput screening.

### DETAILED DESCRIPTION OF THE DRAWINGS

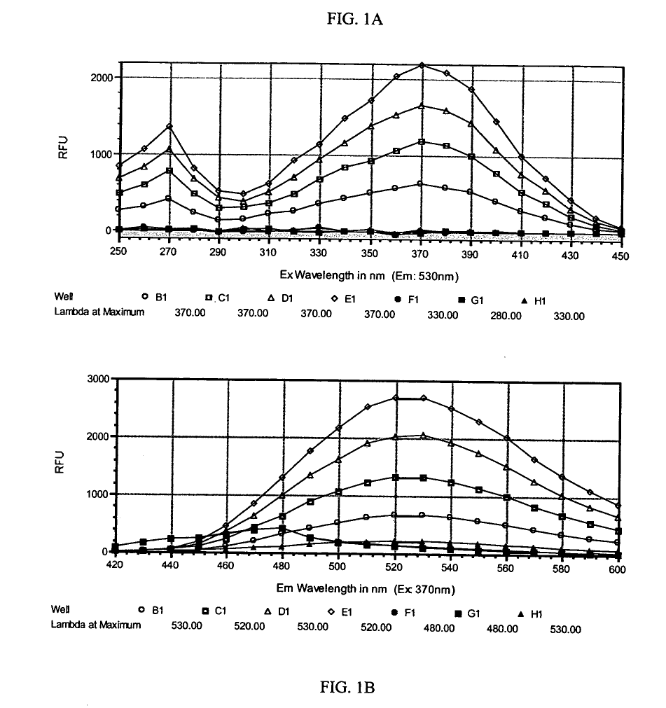

FIG. 1A is a plot of the excitation spectra of the Probe at 2.0 μM (diamonds), 1.5 μM (triangles), 1.0 pM (squares), and 0.5 M (circles) in PBS. Additional spectra shown are baseline scans of PBS.

FIG. 1B is a plot of the emission spectra of the probe at 2.0 μM (diamonds), 1.5 μM (triangles), 1.0 μM (squares), and 0.5 μM (circles) in PBS. Additional spectra shown are baseline scans of PBS.

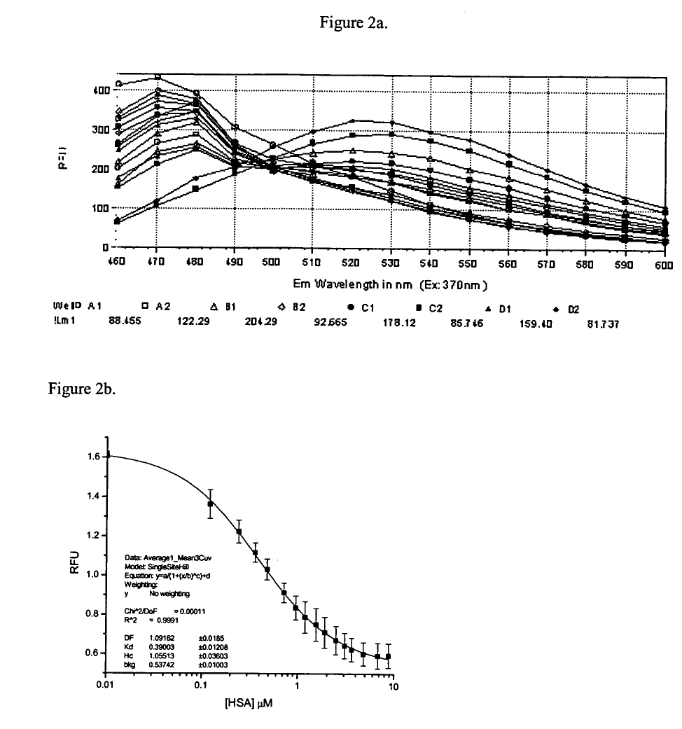

FIG. 2a. is a plot of fluorescence emission spectra of 0.5 μM probe titrated with HSA (V), 0.17 μM HSA per aliquot, in PBS at 23° C. The spectrum with the greatest intensity at 530 nm is the probe in buffer. Spectral intensity at 530 nm decreases with each injection of HSA until saturation.

FIG. 2b. is a plot of data points representing the average binding isotherm from triplicate titrations of 10 nM THE PROBE with HSA(V) in PBS, pH 7.0, error bars indicate the standard deviations. The solid line resulted from least-squares curve fitting the data using the indicated equation for a single site binding isotherm.

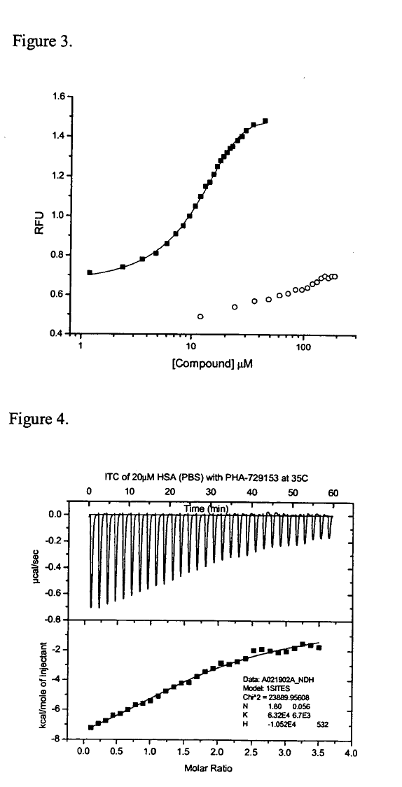

FIG. 3 is a plot of the competitive displacement of the probe from 19 μM HSA by phenylbutazone (circles) or ibuprofen (squares).

FIG. 4 shows two plots. The bottom plot represents the isothermal titrating calorimetry results integrated enthalpies and least-squares curve fit for the probe interaction with HSA (V) in PBS. The top plot shows the enthalpy change per 6 μL injection of a 0.5 mM solution of the probe into a 20 μM solution of HSA (V) in PBS at 35° C.

### DETAILED DESCRIPTION OF THE INVENTION

Definitions

Abbreviations used: HSA, human serum albumin; dansyl-, 5-(dimethylamino)-1-naphthalenesulfonyl-; DNSA, dansyl-1-sulfonamide; DS, dansylsarcosine; PBZ, phenylbutazone; Kd, dissociation constant; PBS, phosphate-buffered saline, MTBE methyl-t-butyl ether; M₂OH methanol; NaOH sodium hydroxide; EtOH ethanol; NBS N-bromo-succinimide; AIBN 2,2′-azobisisobutyronitrile; RT room temperature; THF tetrahydrofuran; DCM dichloromethane;
The term “halo” refers to a halogen atom selected from Cl, Br, I, and F.

The term “alkyl” refers to both straight- and branched-chain moieties. Unless otherwise specifically stated alkyl moieties include between 1 and 9 carbon atoms.

The term “alkenyl” refers to both straight- and branched-chain moieties containing at least one —C═C—. Unless otherwise specifically stated alkenyl moieties include between 1 and 9 carbon atoms.

The term “alkynyl” refers to both straight- and branched-chain moieties containing at least one —C═C—. Unless otherwise specifically stated alkynyl moieties include between 1 and 9 carbon atoms. between 1 and 6 carbon atoms.

The term “alkoxy” refers to —O-alkyl groups.

The term “cycloalkyl” refers to a cyclic alkyl moiety. Unless otherwise specifically stated cycloalkyl moieties will include between 3 and 9 carbon atoms.

The term “cycloalkenyl” refers to a cyclic alkenyl moiety. Unless otherwise specifically stated cycloalkenyl moieties will include between 5 and 9 carbon atoms and at least one —C═C— group within the cyclic ring.

The term “amino” refers to —NH₂.

The term “sulfonamide” refers to a —S(O)₂—N(Q₁₀)₂.

The term “aryl” refers to phenyl and naphthyl.

The term “het” refers to mono- or bi-cyclic ring systems containing at least one heteroatom selected from O, S, and N. Each mono-cyclic ring may be aromatic, saturated, or partially unsaturated. A bi-cyclic ring system may include a mono-cyclic ring containing at least one heteroatom fused with an cycloalkyl or aryl group. A bi-cyclic ring system may also include a mono-cyclic ring containing at least one heteroatom fused with another het, mono-cyclic ring system.

Examples of “het” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

The term “heteroaryl” refers to a mono- or bicylic het in which at least one cyclic ring is aromatic.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀, —S(O)Q₁₀, —OS(O)₂Q₁₀, —C(═NQ₁₀)Q₁₀, —C(═N—O-Q₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(S)NQ₁₀Q₁₀, —N(Q₁₀)C(S)NQ₁₀Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(S)Q₁₀, —NQ₁₀C(O)NQ₁₀Q₁₀, —NQ₁₀C(O)OQ₁₀, —OC(O)NQ₁₀Q₁₀, —NQ₁₀C(S)OQ₁₀, —O—C(S)NQ₁₀Q₁₀, S(O)₂NQ₁₀Q₁₀, —NQ₁₀S(O)₂Q₁₀, —NQ₁₀S(O)Q₁₀, —NQ₁₀SQ₁₀, —NO₂, and —SNQ₁₀Q₁₀. Each of the het, cycloalkyl, cycloalkenyl, and aryl being optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)Q₁₀)₂, —S(O)₂—N═S(Q₁₀)₂, —NQ₁₀Q₁₀, —C(O)Q₁₀, —NQ₁₀C(O)OQ₁₀, —OC(O)NQ₁₀Q₁₀, —NQ₁₀C(S)OQ₁₀, —O—C(S)NQ₁₀Q₁₀—C(S)Q₁₀, —C(O)OQ₁₀, —OC(O)Q₁₀, —C(O)NQ₁₀Q₁₀, —C(S)NQ₁₀Q₁₀—C(O)C(Q₁₆)₂OC(O)Q₁₀, —CN, —NQ₁₀C(O)Q₁₀, —N(Q₁₀)C(S)NQ₁₀Q₁₀, —N(Q₁₀)C(S)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, alkenyl, alkynyl, het, halo, cycloalkyl, cycloalkenyl, and aryl. The het, cycloalkyl, cycloalkenyl, alkenyl, alkynyl, and aryl being optionally substituted with 1-3 substitutuents selected from halo and Q₁₅.

The term “substituted het” includes, but is not limited to, substituted indolyl, and refers to a het moiety including 1-4 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, NQ₁₀C(O)OQ₁₀, —OC(O)NQ₁₀Q₁₀, —NQ₁₀C(S)OQ₁₀, —O—C(S)NQ₁₀Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀OSQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and The term “substituted alkenyl” refers to a alkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═SQ₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —OC(O)—NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted cycloalkenyl” refers to a cycloalkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQIoS(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substituents selected from halo and Q₁₅.

Each Q₁₀ is independently selected from —H, alkyl, cycloalkyl, het, cycloalkenyl, and aryl. The het, alkyl, cycloalkyl, cycloalkenyl, and aryl being optionally substituted with 1-3 substituents selected from halo and Q₁₃.

Each Q₁₃ is independently selected from —H, halo, alkyl, aryl, cycloalkyl, and het. The alkyl, aryl, cycloalkyl, and het being optionally substituted with 1-3 substituents independently selected from halo, —NO₂, —CN, ═S, =0, and Q₁₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —S(O)₂—N═S(O)(Q₁₁)₂, —S(O)₂—N═S(Q₁₁)₂, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —OC(O)—NQ₁₀Q₁₀, —C(S)NQ₁₁Q₁₁, —C(O)C(Q₁₆)₂OC(O)Q₁₀, —CN, ═O, ═S, —NQ₁₁C(O)Q₁₁, —NQ₁₁C(S)Q₁₁, —NQ₁₁C(O)NQ₁₁Q₁₁, —NQ₁₁C(S)NQ₁₁Q₁₁, —S(O)₂NQ₁₁Q₁₁, —NQ₁₁(O)₂Q₁₁, —NQ₁₁S(O)Q₁₁, —NQ₁₁SQ₁₁, —NO₂, and —SNQ₁₁Q₁₁.

Each Q₁₄ is —H or a substituent selected from alkyl, cycloalkyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —C(S)NQ₁₆Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, and —NQ₁₆S(O)₂Q₁₆. The alkyl, cycloalkyl, and cycloalkenyl being further optionally substituted with ═O or ═S.

Each Q₁₅ is alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —OC(O)—NQ₁₀Q₁₀, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S.

Each Q₁₆ is independently selected from —H, alkyl, and cycloalkyl. The alkyl and cycloalkyl optionally including 1-3 halos.

Each Q₁₇ is independently selected from —H, —OH, and alkyl optionally including 1-3 halos and —OH.

It is to be understood that the present invention encompasses any racemic, optically-active, polymorphic, tautomeric, or stereoisomeric form, or mixture thereof, of a compound of the invention, which possesses the useful properties described herein.

The term “optionally substituted” refers to a moiety which may be substituted by one or more groups as defined herein. For example, optionally substituted indolyl refers to an indolyl (a het) that optionally includes one or more groups described above, with respect to the phrase substituted het.

### DETAILED DESCRIPTION

An objective of this invention is to provide a fluorescence based assay for determining compound affinities for human serum albumin. A further objective of this invention is to provide fluorescent probes that have a high quantum yield. Another objective is to provide fluorescent probes that exhibit an excitation wavelength longer than the absorption bands of most other compounds. A still further objective is to provide probes that have a fluorescent emission wavelength in a range with little interference by most other compounds.

This invention describes a method for determining molecular affinities (equilibrium dissociation constants, Kd's or binding constants) of test compounds for human serum albumin (HSA). The molecular affinities are determined by measuring the displacement of a fluorescent compound of Formula I, subsequently referred to as the probe. Affinity of a single compound or high throughput (HT) determinations of compound affinities for HSA may be obtained using this method and a fluorescence reader. HT screening may be performed by measuring sample cells either in a serially, such as one at a time or in a parallel fashion, such as by measuring a plurality of sample cells concurrently.

The probe exhibits useful fluorescent properties such as high quantum yield and a fluorescence emission wavelength maximum of 480-580 nm and an excitation wavelength of 310-430 nm. The excitation wavelength range of the probe is longer than many common drug molecules and thereby reduces problematic inner-filter or compound absorbance effects.

Titration of the probe with HSA indicated the probe binds to HSA and is environmentally sensitive with quenching of the emission at 530 nm observed as the probe binds to HSA. Probe displacement experiments using ligands (ibuprofen, phenylbutazone, warfarin and diclofenac) that bind at known sites on HSA indicated the probe could be displaced by compounds that interact with the Site I and Site II. Further confirmation that the probe binds to multiple sites on HSA was indicated by a stoichiometry of two molecules of probe bound to each molecule of HSA as determined by isothermal titrating calorimetry experiments.

To determine binding constants of molecules using this method, a solution of the probe in buffered water is added to a cuvette or to wells in a multi-well plate, or to another suitable sample cell that can be placed in a fluorescence reader. Fluorescence of the probe solution is measured, a molar excess of human serum albumin is added and the change in fluorescence is recorded. The test compound is then added to the cuvette and equilibrated for 30-60 min at room temperature. The fluorescence is again read and the percentage of probe competitively displaced from HSA calculated by the following equation:
Percent probe displaced=100−[(f₀−fₐ)/(f₀— f_{b})]×100:

Where, f₀ is the fluorescence of the probe in buffer,
f_{b} is the fluorescence of the probe in HSA and
fₐ is the fluorescence of compound, HSA and probe.

The percent probe displaced provides a quantitative measure of the test compounds affinity for the two common drug binding sites on HSA.

An overall equilibrium dissociation constant, K_{d}, can be calculated from the single point displacement values by the equation:
K_{d}=(f−f_{b})/(f₀−f)*[Iₒ−B₀*(f₀−f)/(f₀−f_{b})];

Where, I₀ is the test compound concentration and B₀ is the HSA concentration.

The fluorescence may be measured one sample at a time or multiple samples may be measured simultaneously.

### EXAMPLES

Without further elaboration, it is believed that one skilled in the art can, using the preceding description, practice the present invention to its fullest extent. The following detailed examples describe how to perform the various processes of the invention and are to be construed as merely illustrative, and not limitations of the preceding disclosure in any way whatsoever. The compound referred to as the “probe” in the following examples is 5-cyano-2-[(E)-2-(1-methyl-1H-indol-2-yl)ethenyl]benzoic acid. Those skilled in the art will promptly recognize appropriate variations from the procedures both as to suitable probes and as to analysis conditions and techniques.
Synthesis of the Probe and Analogs Thereof 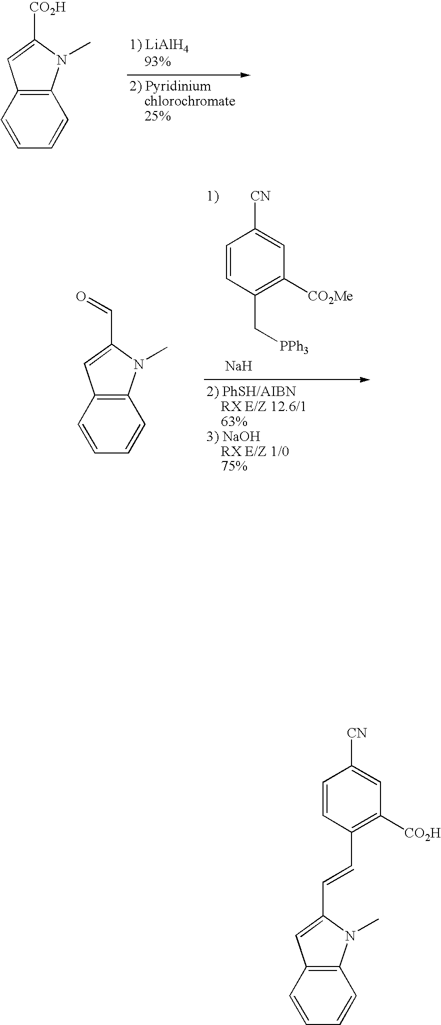
Methyl 2-methyl-5-nitrobenzoate 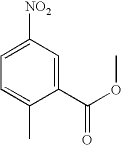
2-Methyl-5-nitrobenzoate (5.0 g, 27.6 mmol) was dissolved in MeOH (0.4 L) followed by the addition of H₂SO₄ (7 mL). The mixture was heated at reflux for 36 h, then cooled to rt and concentrated to ca 100 mL. The solution was diluted with MTBE neutralized with 6N NaOH, washed with 1N NaOH, brine, dried (MgSO₄), filtered and concentrated in vacuo to afford 4.72 g (87%) of methyl 2-methyl-5-nitrobenzoate as a white solid.
Analytical Data for Methyl 2-methyl-5-nitrobenzoate

¹H NMR (, CDCl₃) δ 8.80, 8.25, 7.44, 3.97, 2.74.
Methyl 5-amino-2-methylbenzoate 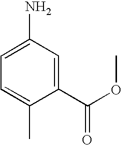
Methyl 2-methyl-5-nitrobenzoate (5.0 g, 25.6 mmol) was dissolved in EtOH with Raney nickel under a 35 psi atmosphere of H₂. The reaction was stirred for 20 h, then filtered through Celite washed with MeOH and concentrated in vacuo to afford 4.2 g (100%) of methyl 5-amino-2-methylbenzoate.
Analytical Data for methyl 5-amino-2-methylbenzoate

¹H NMR, CDCl₃) δ 7.32, 7.06, 6.82, 3.89, 2.49.
Methyl 5-cyano-2-methylbenzoate,

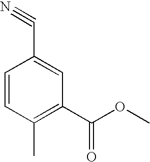

Methyl 5-amino-2-methylbenzoate (4.2 g, 25.4 mmol) was dissolved in MeOH/H₂O (20 mL/46 mL) was cooled with icebath followed by the addition of HCl (54 mL), NaNO₂ (2.63 g, 38.1 mmol, in H₂O 60 mL). The mixture was stirred for 1 h, then neutralized with solid NaHCO₃, extensive gas evolution. Then a cold mixture of KCN (2.48 g, 38 mmol) and CuCN (2.9 g, 33 mmol) in a H₂O (40 ml)/EtOAc (80 mL) was added. The reaction was stirred for ½ h, then filtered through Celite, extracted with EtOAc then washed with H₂O, brine, dried (MgSO₄), filtered and concentrated in vacuo. The residue was purified by silica gel chromatography (heptane/DCM 19/1, 9/1, 1/1, 1/0) to afford 1.89 g (42%) of methyl-5-cyano-2-methylbenzoate as a white solid. Analvtical data for methyl-5-cyano-2-methylbenzoate

¹H NMR, CDCl₃) δ 8.23, 7.68, 7.38, 3.94.
Methyl 2-(bromomethyl)-5-cyanobenzoate

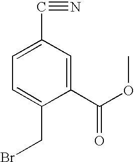

Methyl 5-cyanobenzoate (4.50 g, 25.6 mmol), NBS (5.03 g, 28.25 mmol) and AIBN (150 mg) were dissolved in dichloroethane (160 mL). The mixture was irradiated with a photolamp for 2 h. The mixture was cooled to RT and concentrated in vacuo. The residue was purified by silica gel chromatography (DCM/heptane 1/9, 1/₄, 1/1, 1/0) to afford 4.79 g (73%) of methyl 2-(bromomethyl)-5-cyanobenzoate.

Analytical Data for Methyl 2-(bromomethyl)-5-cyanobenzoate

¹H NMR, CDCl₃) δ 8.29, 7.79, 7.63, 4.97, 4.00.
Methyl 2-{[bromo(triphenyl)phosphoranyl]methyl}-5-cyanobenzoate

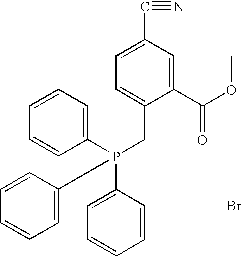

Methyl 2-(bromomethyl)-5-cyanobenzoate (2.80 g, 10.9 mol) was added to a solution of triphenylphosphine (2.87 g, 10.9 mmol) in toluene (50 mL). The resulting mixture was heated at reflux for 3 h, cooled to RT, the precipitate was isolated by filtration, washed with pentane to afford 4.64 g (82%) of methyl 2-{[bromo(triphenyl)phosphoranyl]methyl}-5-cyanobenzoate as a white solid.

Analytical Data for Methyl 2-1-{[bromo(triphenyl)phosphoranyl]methyl}-5-cyanobenzoate

¹H NMR, DMSO-d₆) δ 8.22, 8.08, 8.79-7.51, 5.63, 3.48.
(1-Methyl-1H-indol-2-yl)methanol

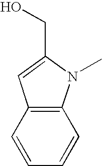

1-Methyl-1H-indole-2-carboxylate (1.0 g, 5.71 mmol) was added to a LiAlH4 (217 mg, 5.71 mmol) in THF (50 mL). Gas evolution was observed and the reaction was stirred at RT for 6 h, then quenched with H₂O (300 μL) then 6N NaOH (300 μL) followed by H₂O (600 μL). The mixture was stirred at RT for 20 min, then filtered through celite and concentrated in vacuo to afford 855 mg (93%) of (1-methyl-1H-indol-2-yl)methanol.

Analytical Data for (1-methyl-1H-indol-2-yl)methanol

¹H NMR, DMSO-d₆) δ 7.49, 7.40, 7.14-6.96, 6.34, 5.22, 4.63, 3.73.
1-Methyl-1H-indole-2-carbaldehyde

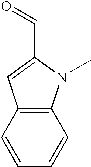

(1-Methyl-1H-indol-2-yl)methanol (850 mg, 5.2 mmol) was dissolved in DCM (100 mL) followed by the addition of pyridinium chlorochromate (2.3 g, 10.5 mmol). The mixture was stirred at RT for 4 h, then poured through a silica plug eluting with DCM. The solvent was evaporated in vacuo and the residue was purified by silica gel chromatography (DCM/heptane/MeOH 1/1/0, 1/0/0, 19/0/1) to afford 216 mg (25%) of 1-methyl-1H-indole-2-carbaldehyde.

Analytical Data for 1-methyl-1H-indole-2-carbaldehyde.

¹H NMR CDCl₃) δ 9.90, 7.75, 7.48-7.41, 7.28, 7.23-7.17, 4.12.
Methyl 5-cyano-2-[(E)-2-(1-methyl-1H-indol-2-yl)ethenyl]benzoate

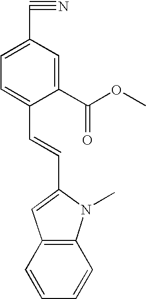

Methyl 2-{[bromo(triphenyl)phosphoranyl]methyl}-5-cyanobenzoate (842 mg, 1.63 mmol) in DMSO (20 mL) followed by the addition of NaH (66 mg, 1.63 mmol). Gas evolution was observed, the reaction was heated to 60° C. for 2 h, then cooled to RT and 1-methyl-1H-indole-2-carbaldehyde (216 mg, 1.3 mmol) was added and the reaction was stirred at RT for 2 h. The mixture was diluted with MTBE, washed with H₂O, brine, dried (MgSO₄), filtered and concentrated in vacuo. The residue was purified by silica gel plug (DCM) to afford a Z/E mixture. The solid was dissolved in toluene (50 mL) followed by the addition of thiophenol (100 μL) and AIBN (14 mg). The reaction was heated at reflux for 12 h, then concentrated in vacuo. The residue was recrystallized from MeOH to afford 272 mg (63%) of 5-cyano-2-[(E)-2-(1-methyl-1H-indol-2-yl)ethenyl]benzoate as a E/Z (12.6/1) mixture.

Analytical Data for Methyl 5-cyano-2-[(E)-2-(1-methyl-1H-indol-2-yl)ethenyl]benzoate

¹H NMR, CDCl₃) δ 8.27, 8.11, 7.88 (D, J=8.3 Hz, 1H), 7.77 (dd, J=1.7, 8.3 Hz, 1H), 7.63, 7.35-7.11, 3.69, 3.88.
5-Cyano-2-[(E)-2-(1-methyl-1H-indol-2-yl)ethenyl]benzoic Acid

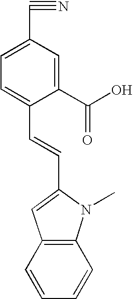

Methyl 5-cyano-2-[(E)-2-(1-methyl-1H-indol-2-yl)ethenyl]benzoate (272 mg, 0.86 mmol) was dissolved in THF (50 mL) and 6N NaOH (5 mL) was added, the resulting mixture was stirred for 48 at rt, then diluted with MTBE, washed with 1N HCl, H₂O, brine, dried (MgSO₄) filtered and concentrated in vacuo. The residue was recrystallized from MeOH to afford 197 mg (75%) of as a pure E-only isomer.

Analytical data for

¹H NMR (DMSO-d₆) δ 13.70, 8.24-8.21, 8.03-8.00, 7.99, 7.60, 7.56, 7.47, 7.19-7.15, 7.04, 6.87, 3.88.

### Example 1

### General Procedure

Buffered probe solution (200 μL of 0.5 μM probe in phosphate buffered saline) was added to each well of a 96 well plate using the automated liquid injection device on the instrument. To each well, except wells in the last row, was added 15 μL of 20 μM HSA. Test compounds were added to wells, except the last two rows of the plate, equilibrated for 60 minutes at room temperature and then the fluorescence was read for the test compound wells (f), the row of wells containing probe, buffer and HSA (f₀), and the last row of wells containing only probe and buffer (f_{b}). Precision in the fluorescence measurements was determined to be within 3-6% and assay reproducibility over an extended period of one month was within 10% using multiple preparations of HSA, probe solution and stock test compound.

### Example 2

### Chemicals, Equipment and Assay Stock Solutions

Human serum albumin, fraction V, with a purity ≧95% as indicated by cellulose acetate electrophoresis was purchased from Calbiochem (catalog no. 12666). Dulbecco's phosphate buffered saline (PBS) without magnesium or calcium was purchased from Gibco. Dimethylsulfoxide was purchased from EM Science. HSA stock solution was prepared in Dulbecco's PBS at a nominal concentration of 5 mg/mL (77 μM). Accurate concentration of the HSA stock solution was determined by measuring the absorbance of 500 μl of stock solution at 280 run with a Perkin Elmer Lambda 40 UV Spectrophotometer, ε₂₈₀=35,600 M⁻¹cm⁻¹. HSA stock solutions were stored at 5° C. A stock solution of THE PROBE, FW 302.3 g/M, was prepared at 10 mM in DMSO, 0.45 mg of THE PROBE in 150 μL DMSO. This solution was stored in an amber bottle at 5° C.

A SPECTRAmax GEMINI dual-scanning microplate spectrofluorometer (Molecular Devices, Sunnyvale, Calif.) was used to collect the fluorescence data and the autoinjector system from a BMG Polarstar Galaxy (BMG Lab Technologies, Offenburg Germany) was used to dispense buffer and albumin into the assay plates. Assay plates from Corning (catalog no. 3915) were black polystyrene, 96 well with flat bottoms.

### Example 3

### The Probe Displacement Assay Procedure

Analyte solutions were prepared at 5 mM in DMSO. Aliquots, 0.5 μL, were dispensed into assay wells in triplicate starting with wells A1, B1 and C1. This resulted in a final analyte concentration of 12 μM after addition of 200 μL of PBS plus THE PROBE and 15 μL of HSA at 20 μM. The next analyte was added to wells D1, E1 and F1. In this manner, 2 analytes per column or 21 compounds in all could be assayed on one 96 well plate. The last two columns of the assay plate were reserved for assay of the standards and the last two rows were reserved for replicate assay of controls and blanks. A typical plate layout is shown in Table 1.

A 5 μl aliquot of THE PROBE was diluted into 100 mL of PBS for a final probe concentration of 0.5 μM. Injector number one of the Polarstar was filled with this solution and 200 μl of the probe solution was added to each well of the assay plate. The plate was transferred to a SpectraMax Gemini reader for data collection. This first read without albumin added was done to determine any effect of analyte on the fluorescence response. Data acquisition was controlled using SOFTmax PRO 3.1.1 software on the SpectraMax Gemini plate reader at a constant temperature of 24° C. The excitation wavelength was 370 nm and the emission wavelength was 533 nm with a 530 nm cut-off filter. Detector response was set to high sensitivity with 14 reads/well. Each compound was assayed in triplicate with the average and standard deviation calculated using SOFTmax Pro software.

Stock HSA solution was diluted to 20 μM by adding 0.60 mL of 77 μM stock to 1.7 mL of PBS. Injector number two of the Polarstar was filled with 20 μM HSA and 15 μl aliquots added to all wells except the blanks resulting in a final HSA concentration of 1.4 μM. The plate was read again using the same parameters after the HSA addition was completed and another read performed after 60 min. incubation at 24° C.

After the plate was read for the final time, the percentage of THE PROBE competitively displaced from HSA was calculated from the average relative fluorescence values by the equation:
D=100−[(f₀−fₐ)/(f₀−f_{b})]*100,
where, D is the percent THE PROBE displaced from HSA by analyte, f₀ is the fluorescence of THE PROBE in PBS, f_{b} is the fluorescence of THE PROBE bound to HSA, and fₐ is the fluorescence with analyte added.

### Example 4

### Displacement of THE PROBE by Compounds Known to bind at Site I and Site II

Following the assay protocol of Example 3, compounds known to bind HSA at site I or site II were examined for their abilities to displace THE PROBE from HSA.

The compounds used included, ibuprofen with high affinity for site II, warfarin and phenylbutazone with high affinity for site I and diclofenac with affinity for both sites. The percent of THE PROBE displaced by each of the compounds is shown in Table 2.

### Example 5

### Isothermal Titration Calorimetry

Isothermal titration calorimetry experiments were performed using an OMEGA titrating microcalorimeter from Microcal, Inc. (Northampton, Mass.). Data collection, analysis, and plotting were performed using a Windows-based software package (Origin 5.0) supplied by the instrument vendor. The titrating microcalorimeter consisted of a sample and reference cell held in an adiabatic enclosure. The calorimeter was calibrated by comparing the measured areas of applied heat pulses to known values. Known and experimentally measured values agreed to within 2%.

The probe was prepared in DMSO at 10 mM and diluted to 500 μM in PBS. the probe and HSA solutions were degassed prior to analysis. The reference cell was filled with PBS. A 20 μM solution of HSA in PBS was placed in the 1.37 mL sample cell and the probe (0.5 mM) was held in a 250 μL syringe. Thirty injections (6 μL each) of the probe were made by a computer-controlled stepper motor into the sample cell filled with HSA held at 37° C. The syringe stir rate was 400 rpm. The heat adsorbed or released with each injection was measured by a thermoelectric device connected to a Microcal nanovolt preamplifier. Titration isotherms for the binding interactions were comprised of the differential heat flow for each injection. Heats of dilution obtained by injecting ligand into dialysate buffer were minor but were subtracted prior to fitting the data. Isotherms fit well to a single site model (Wiseman, T., et. al. Anal. Biochem (1989) 179, 131-137) using an iterative nonlinear least-squares algorithm included with the instrument.

All parameters were floated during the iterations until a minimum %² was obtained between experimental and fit data. Deconvolution of the isotherms by this method provided the binding constant, K, change in enthalpy, ΔH, and stoichiometry, N, of binding for each interaction. The change in free energy (ΔG) and change in entropy (ΔS) were determined using the Gibbs' free energy equation.

### Example 6

### Characterization of the Probe and its Interaction With HSA

FIG. 1 shows the excitation and emission spectra for the probe. The excitation maximum was at 370 nm and the emission maximum was at 533 nm. A linear response in intensity versus concentration of the probe was detected over the range examined. Titration of 0.5 μM the probe with HSA (fraction V) indicated the fluorescence emission intensity of the probe was environmentally sensitive. As shown in FIG. 2a, fluorescence intensity of the probe at 533 nm decreased with increasing concentration of HSA and there was a corresponding increase in intensity at 470 nm. Fractional saturation of the HSA binding site(s) for the probe can therefore be determined by following the change in fluorescence of the probe as it binds or is released from HSA. FIG. 2b shows the average binding isotherm obtained for the interaction of the probe with HSA. The data represents the average isotherm from triplicate titrations of 110 nM the probe with HSA in PBS at 23° C. These data were well fit using an equation for a single site binding isotherm resulting in an equilibrium dissociation constant, K_{d}, of 0.39 μM. Although the data fit a single site model, the probe could be displaced by compounds known to bind at either site I or site II on HSA (Sudlow, et.al, Mol. Pharm. 11, 824 (1975)). Table 2 indicated that 12 PM ibuprofen, warfarin, diclofenac and phenylbutazone were each able to displace the probe from HSA. Ibuprofen has an affinity at site II of 1 μM (Peters, All about Albumin, Academic Press, New York, 432(1996)). Warfarin and phenylbutazone bound at site I with K_{d}'s of 3 and 1.4 μM, respectively (Peters, 1996). High affinity binding (K_{d}=2 μM) of diclofenac was suggested to occur at the benzodiazepine site with the second site (K_{d}=17 μM) shared with warfarin (Chamouard et.al. Biochem. Pharm. 34 (10), 1695, (1985)).

In addition to the single concentration displacement experiments, displacement of the probe from HSA was examined by titrating a solution of the probe bound to HSA with ibuprofen or phenylbutazone. FIG. 3 shows the result of that experiment where the starting concentration of the probe was 0.5 μM with 19 μM HSA in PBS. Ibuprofen was able to displace about 75-80% of the probe bound to HSA and most of the remaining probe bound could be displaced by titration with phenylbutazone.

FIG. 3 shows the binding isotherm for the interaction of the probe with HSA as measured by isothermal titrating calorimetry (ITC). The top panel shows the heat flow per 6 μL injection of the probe into HSA. The heat of dilution of the probe into buffer was negligible. The bottom panel of FIG. 3 shows the integrated heats for each injection and the best fit line from the single-site model used. The agreement between the model and the data was good and indicated approximately 2 molecules of the probe bound per HSA. The enthalpy change for the interaction was −10.4 kcal/M with an average affinity for all binding interactions of 15 μM.

### Example 7

### HSA Affinity Determined by Competitive Displacement of the Probe

Compounds having antimicrobial activity in an analog series were examined using the competitive displacement assay described in Example 3. The percentage displacement of the probe from HSA for each of the compounds is listed in Table 3. Table 3 also lists the ratio of MIC values vs. S. aureus 9218 for each compound with and without 10% serum. There was a linear relationship between the percentage displacement values and the MIC ratio with a correlation of 0.67. This indicated the affinity of the compound for HSA was directly related to serum's effect on MIC for this set of antimicrobial compounds.

TABLE 1
Plate Layout For the probe/HSA Displacement Assay

| | | | | | | | | | | | |
|---|---|---|---|---|---|---|---|---|---|---|---|
| C1^{a} | C3 | C5 | C7 | C9 | C11 | C14 | C16 | C18 | C20 | S1^{b} | S3 |
| C1 | C3 | C5 | C7 | C9 | C11 | C14 | C16 | C18 | C20 | S1 | S3 |
| C1 | C3 | C5 | C7 | C9 | C12 | C14 | C16 | C18 | C20 | S1 | S3 |
| C2 | C4 | C6 | C8 | C10 | C13 | C15 | C17 | C19 | C21 | S2 | S4 |
| C2 | C4 | C6 | C8 | C10 | C13 | C15 | C17 | C19 | C21 | S2 | S4 |
| C2 | C4 | C6 | C8 | C10 | C13 | C15 | C17 | C19 | C21 | S2 | S4 |
| HSA/probe^{c} | | | | | | | | | | | |
| probe^{d} | | | | | | | | | | | |

^{a}C# indicates analyte #1
^{b}S# indicates standard #1
^{c}1.4 μM HSA + .5 μM the probe in first three columns of this row
^{d}0.5 μM the probe in first three columns of this row

TABLE 2
Displacement of 0.5 μM the probe from 1.4 μM HSA
by known ligands.

% Displaced

| Compound | Trial #1 | Trial #2 |
|---|---|---|
| Ibuprofen | 47 | 54 |
| Warfarin | 11 | 15 |
| Phenylbutazone | 24 | 39 |
| Diclofenac | 41 | 45 |

TABLE 3
Comparison of Percent the probe Displaced from HSA by Compound
Compared to the Effect of Serum on S. aureus 9218 MIC.

| | MIC vs. SA | |
|---|---|---|
| | 9218(with | % Displacement of |
| | 10% Serum) | the probe from |
| | over SA | HSA by 12 μM |
| Compound No. | 9218 | compound |
| 1 | 64 | 56.7 |
| 2 | 8 | 4.8 |
| 3 | 16 | 25.4 |
| 4 | 64 | 41.1 |
| 5 | 64 | 63.2 |
| 6 | 32 | 19.1 |
| 7 | 16 | 26.3 |
| 8 | 32 | 69.3 |
| 9 | 64 | 75.7 |
| 10 | 32 | 69.3 |
| 11 | 16 | 43.1 |
| 12 | 64 | 73.3 |
| 13 | 8 | 16.7 |
| 14 | 32 | 51.6 |
| 15 | 128 | 98.8 |
| 16 | 8 | 33.8 |
| 17 | 8 | 17 |
| 18 | 4 | 14 |

1. A method for determining the binding affinity of an analyte comprising the steps of:
a) providing a buffer solution;
b) adding an analyte to the solution;
c) adding a probe compound which binds a plurality of Human Serum Albumin sites to the solution;
d) adding Human Serum Albumin to the solution;
e) irradiating the solution containing the analyte, probe compound, and Human Serum Albumin;
f) measuring the fluorescence of the irradiated solution; and
g) calculating the binding affinity of the analyte based on the measured fluorescence.

2. The method of claim 1, wherein the probe is a compound of Formula I 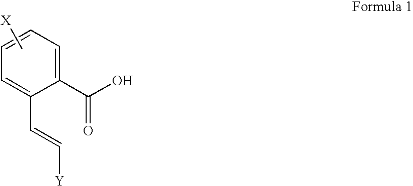
wherein:
X is one to three substituents selected from the group of halogen, —CN, NO₂, aryl, —C(O)—R, in which R is an optionally substituted C1-C4 alkyl or an optionally substituted aryl; and
Y is substituted or unsubstituted heteroaryl.

3. The method of claim 2, wherein Y is an optionally substituted indolyl.

4. The method of claim 3, wherein Y is 1-methyl-indol-2-yl.

5. The method of claim 2, wherein the probe is 5-cyano-2-[(E)-2-(1-methyl-1H-indol-2-yl)ethenyl]benzoic acid.

6. The method of claim 1, wherein calculating the binding affinity of the analyte includes determining the percent displacement of the probe via the equation
D=100−[(f₀−fₐ)/(f₀−f_{b})]*100,
where, D is the percent THE PROBE displaced from Human Serum Albumin by the analyte, f₀ is the fluorescence of THE PROBE in the buffer solution, f_{b} is the fluorescence of THE PROBE bound to HSA, and fₐ is the fluorescence of the solution containing the analyte and the probe compound.

7. The method of claim 1, wherein the fluorescence is measured between 480-580 nm.

8. The method of claim 1, further comprising measuring the fluorescence of the solution containing the probe compound and the analyte.

9. A method of conducting a high-throughput screen, comprising the steps of
a) providing a plurality of buffer solutions;
b) providing a plurality of analytes;
c) adding one analyte from the plurality to a plurality of the buffer solutions;
d) adding a probe compound which binds to sites I and II of Human Serum Albumin to a plurality of the solutions containing an analyte;
e) adding Human Serum Albumin to a plurality of the solutions containing an analyte, and the probe compound;
f) irradiating a plurality of the solutions containing the analyte, the probe compound, and Human Serum Albumin;
g) measuring the fluorescence of the irradiated solutions; and
h) calculating the binding affinity of the analyte in each of the solutions based on the measured fluorescence.

10. The method of claim 9, wherein the probe is a compound of Formula I 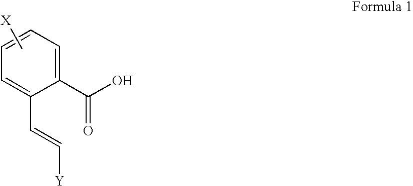
wherein:
X is one to three substituents selected from the group of halogen, —CN, NO₂, aryl, —C(O)—R, in which R is an optionally substituted C1-C4 alkyl or an optionally substituted aryl; and
Y is substituted or unsubstituted heteroaryl.

11. The method of claim 10, wherein Y is an optionally substituted indolyl.

12. The method of claim 11, wherein Y is 1-methyl-indol-2-yl.

13. The method of claim 10, wherein the probe is 5-cyano-2-[(E)-2-(1-methyl-1H-indol-2-yl)ethenyl]benzoic acid.

14. The method of claim 9, wherein calculating the binding affinity of each analyte includes determining the percent displacement of the probe via the equation
D=100−[(f₀−fₐ)/(f₀−f_{b})]*100,
where, D is the percent THE PROBE displaced from Human Serum Albumin by the analyte, f₀ is the fluorescence of THE PROBE in the buffer solution, f_{b} is the fluorescence of THE PROBE bound to HSA, and fₐ is the fluorescence of the solution containing the analyte and the probe compound.

15. The method of claim 9, wherein the fluorescence is measured between 480-580 nm.

16. The method of claim 9, further comprising measuring the fluorescence of a plurality of the solutions containing the probe compound and the analyte.
